(19) United States
(12) Patent Application Publication
(10) Pub. No.: US 2026/0083928 A1
(43) Pub. Date: Mar. 26, 2026

(54) GAS INHALATION MASK

(71) Applicant: NIHON KOHDEN CORPORATION, Tokyo (JP)

(72) Inventors: Fumihiko TAKATORI, Tokorozawa-shi, Saitama (JP); Kenichiro KABUMOTO, Tokorozawa-shi, Saitama (JP); Yuya BABA, Tokorozawa-shi, Saitama (JP)

(73) Assignee: NIHON KOHDEN CORPORATION, Tokyo (JP)

(21) Appl. No.: 19/112,486

(22) PCT Filed: Sep. 20, 2023
(86) PCT No.: PCT/JP2023/034126

§ 371 Date: Mar. 17, 2025

(30) Foreign Application Priority Data
Sep. 30, 2022 (JP) 2022-158571

(51) Int. Cl. A61M 16/06; A61M 16/08; A61M 16/10

(52) U.S. Cl. CPC A61M 16/06; A61M 16/0816; A61M 16/106; A61M 2205/3306; A61M 2205/7536

(57) ABSTRACT

A gas inhalation mask includes a mask body formed of paper, nonwoven fabric, or fabric, and a tube attachment member. The tube attachment member is attached to an attachment opening provided in the mask body. In a case where the mask body is attached to a subject, an axis of a tube does not directly face toward a face of the subject. The tube is connected to a tube connecting portion of the tube attachment member.

### TECHNICAL FIELD

The presently disclosed subject matter relates to a gas inhalation mask for medical use such as an oxygen mask.

### BACKGROUND

In a case where a gas such as oxygen is supplied to a subject such as a patient, a gas inhalation mask is attached to the face of the subject, a gas inhalation mask is connected with a tube, and the gas is supplied from the tube to be inhaled by the subject. Such a gas inhalation mask is entirely formed of plastic. Patent Literature 1 describes a mask apparatus for supplying oxygen to a patient. In the mask apparatus of Patent Literature 1, the mask body attached to cover the nose portion and the mouth portion of the patient is formed of substantially transparent soft vinyl chloride.

### CITATION LIST

### Patent Literature

PTL 1: WO2020/080357A1

### SUMMARY

### Technical Problem

In a gas inhalation mask for medical use such as an oxygen mask, the mask body attached to cover the nose and the mouth of a subject is formed by injection molding or the like, and thus has a thickness to some extent. The weight of plastic such as vinyl chloride for forming the mask body is about 100 g. On the other hand, a gas inhalation mask for medical use such as an oxygen mask is used in contact with a subject, and thus is made disposable. This generates a large amount of plastic waste. Such a situation causes a large environmental burden, and requires improvement from the viewpoint of environmental problems in SDGs (the Sustainable Development Goals). Gas inhalation masks other than those for medical also use a large amount of plastic.

### SOLUTION TO PROBLEM

A gas inhalation mask including:
a mask body formed of paper, nonwoven fabric, or fabric; and
a tube attachment member,
in which the tube attachment member is attached to an attachment opening provided in the mask body, and
in a case where the mask body is attached to a subject, an axis of a tube does not directly face toward a face of the subject, the tube being connected to a tube connecting portion of the tube attachment member.

### EFFECTS OF THE INVENTION

The amount of plastic waste can be reduced by forming the mask body from paper, nonwoven fabric, or fabric. Being formed of paper, nonwoven fabric, or fabric, the mask body has a strength lower than that of plastic. However, since the axis of the tube connected to the tube connecting portion of the tube attachment member is not directly oriented toward the face of the subject, the force that deforms the mask body is unlikely to transmit from the tube to the mask body. Accordingly, it is possible to provide a gas inhalation mask that is environmentally friendly and has sufficient strength.

### BRIEF DESCRIPTION OF DRAWINGS

FIG. 1 is a front view of a gas inhalation mask in Embodiment 1.

FIG. 2 is a side view of the gas inhalation mask in Embodiment 1.

FIG. 3 is a front view of a mask body in Embodiment 1.

FIG. 4 is a front view of a tube attachment member in Embodiment 1.

FIG. 5 is a rear view of the tube attachment member in Embodiment 1.

FIG. 6 is a side view of the tube attachment member in Embodiment 1.

FIG. 7 is a side view of the gas inhalation mask when attached to a subject in Embodiment 1.

FIG. 8 is a front view of a gas inhalation mask in Embodiment 2.

FIG. 9 is a side view of the gas inhalation mask in Embodiment 2.

FIG. 10 is a front view of a tube attachment member according to Embodiment 2.

FIG. 11 is a side view of the tube attachment member in Embodiment 2.

FIG. 12 is a bottom view of the tube attachment member in Embodiment 2.

FIG. 13 is a rear view of the tube attachment member in Embodiment 2.

FIG. 14 is a bottom view of the tube attachment member to which a gas sensor is attached in Embodiment 2.

FIG. 15 is a side view of a gas inhalation mask attached to the subject in Embodiment 3.

FIG. 16 is a rear view of the mask body in Embodiment 3.

FIG. 17 is a front view of a mask body in Embodiment 4.

FIG. 18 is a side view of a gas inhalation mask attached to the subject in Embodiment 5.

FIG. 19 is a side view of a gas inhalation mask in Embodiment 6.

### DESCRIPTION OF EMBODIMENTS

In the present application, in the drawing, the direction toward the forehead in a case where the gas inhalation mask is attached to the subject is described as up, the direction toward the chin in a case where the gas inhalation mask is attached to the subject is described as down, and the directions toward the cheeks in a case where the gas inhalation mask is attached to the subject are described as lateral. A rotating tube attachment member is also described with the vertical direction in the rotational position in the drawings. A direction in which the subject faces in a case where the gas inhalation mask is attached to the subject is referred to as front, and a direction opposite thereto is referred to as rear.

### Embodiment 1

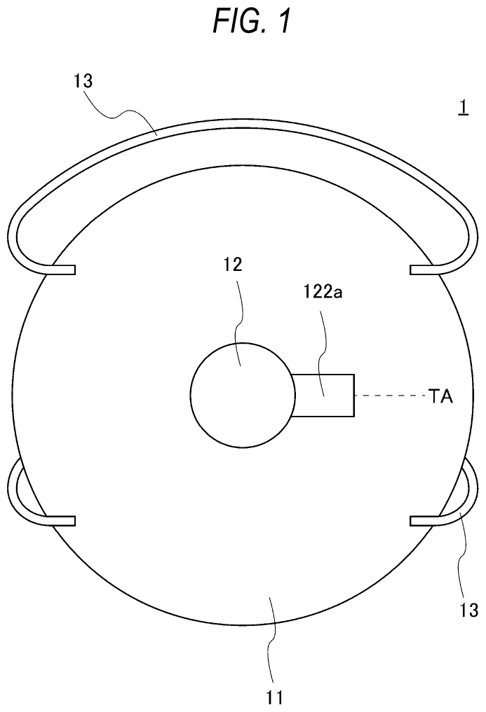

FIG. 1 illustrates a front view of a gas inhalation mask 1 in Embodiment 1. In Embodiment 1, a tube attachment member 12 is attached to the center of a hemispherical mask body 11 to form the gas inhalation mask 1. Two elastic bands 13 are fixed to the mask body 11. The gas inhalation mask 1 is configured to be fixed to the face of a subject S by stretching the two elastic bands 13 around to the back of the head of the subject S.

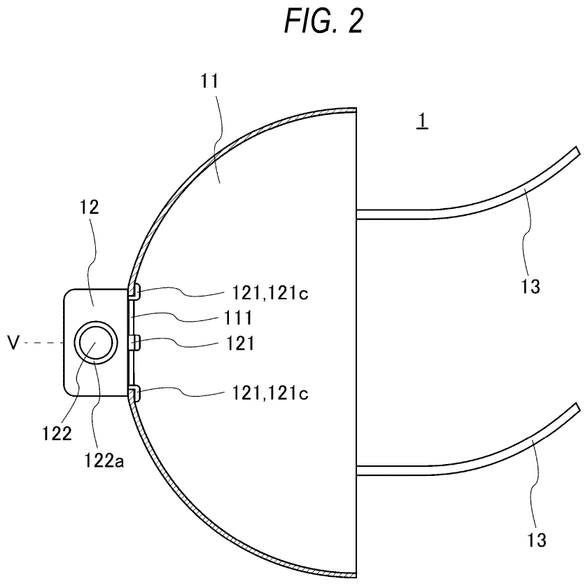

FIG. 2 is a side view of the gas inhalation mask 1 of Embodiment 1. In FIG. 2, the mask body 11 is illustrated in a side cross-sectional view, and the tube attachment member 12 attached to the mask body 11 is illustrated in a side view. The mask body 11 has a hemispherical shape. The tube attachment member 12 may include an inhalation flow path 122 therein. The inhalation flow path 122 passes through the tube attachment member 12 from a tube connecting portion 122a, and communicates with the inside of the mask body 11 from an attachment opening 111 of the mask body 11.

The mask body 11 of Embodiment 1 is formed of thick paper made of natural fiber as a raw material. The tube attachment member 12 is formed of hard vinyl chloride that is hard plastic. Therefore, when the gas inhalation mask 1 is discarded, the mask body 11 and the tube attachment member 12 can be separated and distinguished.

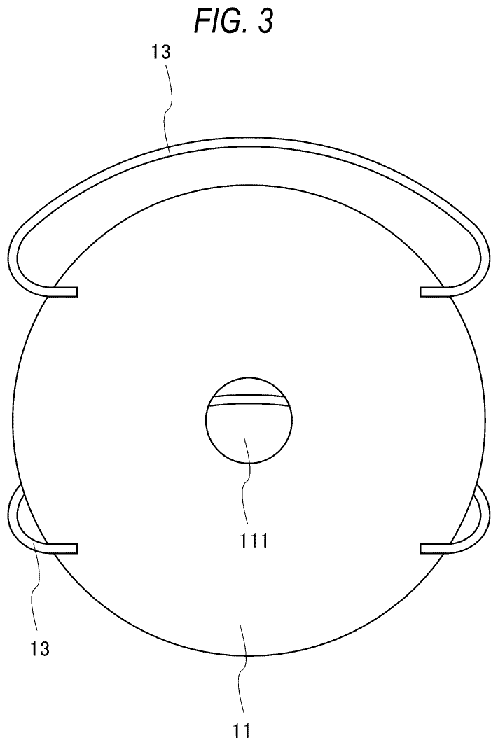

FIG. 3 is a front view of the mask body 11 of the gas inhalation mask 1 illustrated in FIGS. 1 and 2. The center of the mask body 11 is provided with a circular attachment opening 111. In the vicinity of the circumferential end of the mask body 11, two elastic bands 13 are fixed at four positions on the outer surface. The mask body 11 of Embodiment 1 is formed of thick paper made of natural fiber as a raw material. The mask body 11 of Embodiment 1 has strength necessary for the mask body 11 and flexibility to some extent.

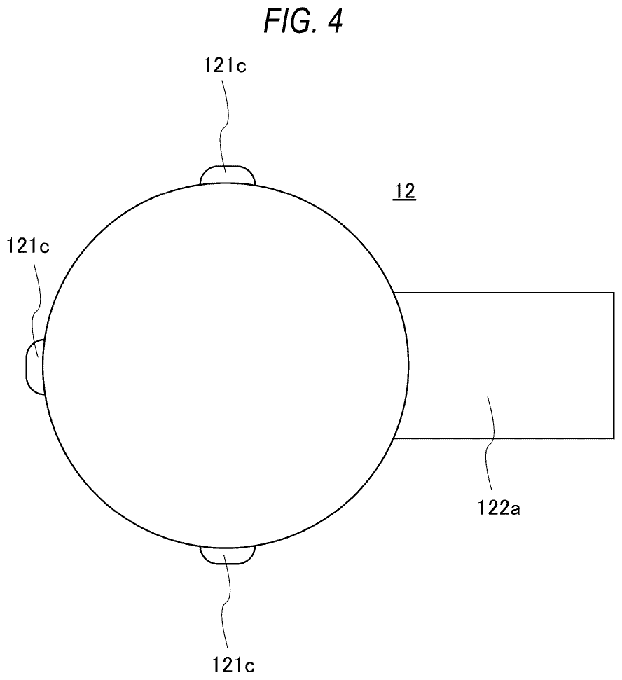

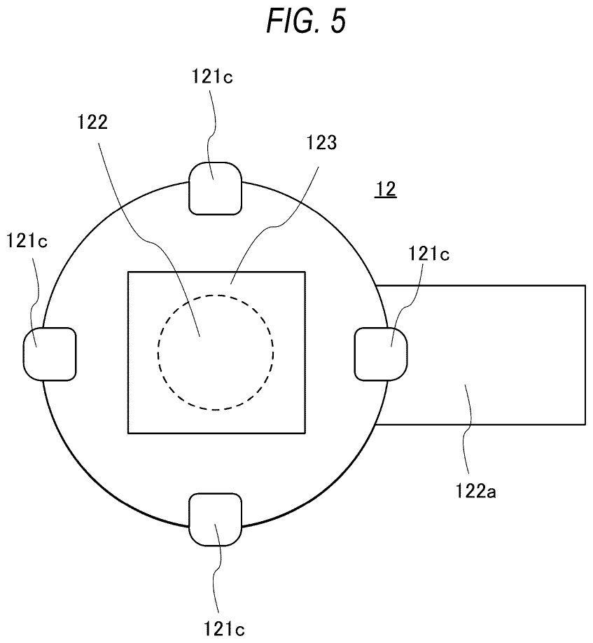

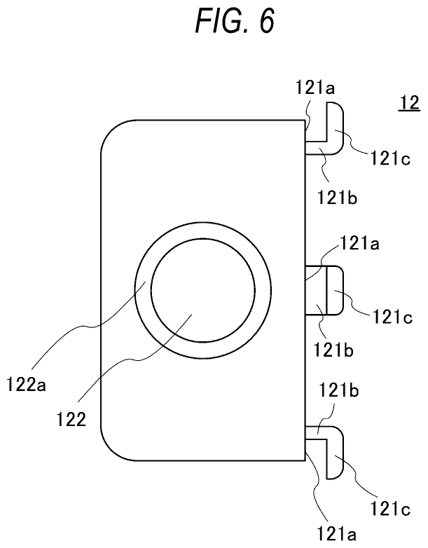

FIGS. 4 to 6 illustrate the tube attachment member 12 illustrated in FIGS. 1 and 2 in an enlarged manner. FIG. 4 illustrates a front view of the tube attachment member 12 of the gas inhalation mask 1. FIG. 5 illustrates a rear view of the tube attachment member 12 of the gas inhalation mask 1. FIG. 6 illustrates a side view of the tube attachment member 12 of the gas inhalation mask 1. In the tube attachment member 12, four locking portion connecting portions 121b protrude rearward from front locking portions 121a on the rear side, which is right side in FIG. 6. The tip of each locking portion connecting portion 121b includes the rear locking portion 121c extending in the vertical direction and the lateral direction of the tube attachment member 12.

As illustrated in FIG. 5, on the rear side of the tube attachment member 12, a hole of the inhalation flow path 122 is covered with a cover 123. The cover 123 is formed from a mesh member. The cover 123 is bonded and fixed around the hole of the inhalation flow path 122 on the rear surface of the tube attachment member 12. Oxygen passes through the inhalation flow path 122 and is then supplied to the rear side of the mask body 11 through the cover 123. On the other hand, the presence of the cover 123 prevents the vomitus or vomited liquid from the mouth of the subject S from entering the inhalation flow path 122. The vomitus does not enter the inhalation flow path 122 even in a case where the supply of oxygen from the inhalation flow path 122 is stopped. Therefore, the vomitus or vomited liquid can be prevented from entering the tube for supplying oxygen. The tube, which is not contaminated by vomitus or the like, does not need to be disposable, and can be reused to further reduce the disposal amount of plastic.

As illustrated in FIG. 6, the front side of each rear locking portion 121c is the surface of each front locking portion 121a. As illustrated in FIG. 2, the tube attachment member 12 is attached to the attachment opening 111 of the mask body 11 such that the rear locking portions 121c are positioned on the rear side of the attachment opening 111. The locking portion connecting portions 121b illustrated in FIG. 6 are positioned inside the circular attachment opening 111 illustrated in FIG. 3. The periphery of the attachment opening 111 of the mask body 11 is sandwiched between the front locking portions 121a and the rear locking portions 121c. Thus, the tube attachment member 12 is attached, to the mask body 11, to be rotatable. In FIG. 1, the tube connecting portion 122a of the tube attachment member 12 protrudes rightward, and the tube axis TA is located on the right, but the direction of the tube axis TA can be rotated in a range of 360°, including leftward, vertical, and the like.

In a case where the tube attachment member 12 is to be attached to the mask body 11, the tube attachment member 12 approaches the attachment opening 111 from the front side of the mask body 11, which is the left side in FIG. 2. The four rear locking portions 121c formed on the tube attachment member 12 are sequentially inserted into the attachment opening 111 of the mask body 11. In a case where the attachment is completed in this manner, the tube attachment member 12 is unlikely to be detached from the mask body 11 because the periphery of the attachment opening 111 is sandwiched between the front locking portions 121a and the rear locking portions 121c at four positions. Further, the locking portion connecting portions 121b are positioned at four positions inside the circular attachment opening 111, the locking portion connecting portions 121b are movable along the inner side of the attachment opening 111, and the tube attachment member 12 is configured to be rotate in a range of 360°. In this manner, the direction in which the tube connecting portion 122a and the tube axis TA illustrated in FIG. 1 face is configured to be rotated in a range of 360°.

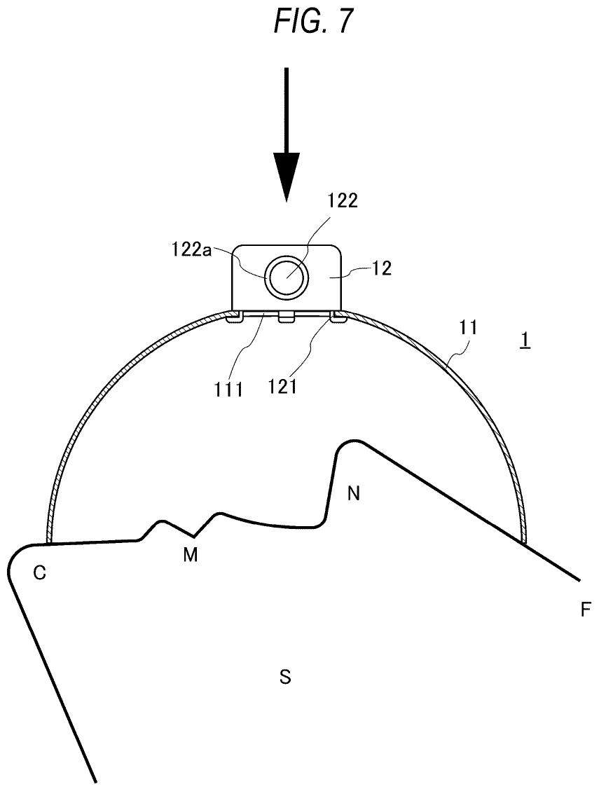

FIG. 7 is a side view illustrating a state in which the gas inhalation mask 1 of Embodiment 1 is attached to the face of the subject S. The mask body 11 is illustrated in a cross-sectional view. In FIG. 7, the gas inhalation mask 1 is attached to the face of the subject S facing upward. The elastic band 13 is not illustrated. As illustrated in FIG. 7, in a case where the mask body 11 is attached to the subject S, the center of the attachment opening 111 is located between the nose N and the mouth M of the subject S, as viewed from the front indicated by the arrow.

In Embodiment 1, the center of the attachment opening 111 coincides with the center of the mask body 11. Although not illustrated, the outlet of the inhalation flow path 122 of the tube attachment member 12 provided at the mask body 11 side is circular. The center of the outlet coincides with the center of the attachment opening 111. The deviation between the center of the mask body 11 and the center of the attachment opening 111 is preferably within 10% of the width of the mask body 11 in both the longitudinal direction and the lateral direction, where the longitudinal direction is defined as a direction from the forehead F to chin C in a case where the gas inhalation mask 1 is attached to the face of the subject S, and the lateral direction is defined as the left-right direction of the face.

As illustrated in FIG. 7, the mask body 11 has a shape that generates a space having a volume to some extent between the mask body 11 and the face of the subject S. In a case where this volume is small, the oxygen supplied into the mask body 11 at the time of exhalation by the subject S leaks out of the mask body 11 without being inhaled by the subject S. However, a volume to some extent allows a part of the oxygen supplied into the mask body 11 at the time of exhalation to stay in the space to be inhaled by the subject S at the time of inhalation. This can obtain good oxygen supply efficiency to the subject S. In a case where the gas inhalation mask 1 is attached to the face of the subject S, the space is preferably 30 cc or more and 200 cc or less, and more preferably 80 cc or more and 150 cc or less.

As understood from FIGS. 1 and 6, in the tube connecting portion 122a, a tube axis TA indicated by a dotted line in FIG. 1, which is the axis of the tube to be attached (not illustrated), is in a lateral direction with respect to the face. The tube axis TA is in a direction of 90° with respect to the vertical direction V, which is indicated by the dotted line in FIG. 2, of the attachment opening 111. Accordingly, the tube is configured to be connected, to the tube connecting portion 122a, in a direction shifted from the vertical direction V of the attachment opening 111. Further, according to the gas inhalation mask 1 of Embodiment 1, the tube axis TA does not directly face toward the face of the subject S in a case where the mask body 11 is attached to the face of the subject S. Therefore, the force for deforming the mask body 11 is unlikely to transmit from the tube to the mask body 11. Further, the tube can be routed easily in a case where the gas inhalation mask 1 is attached to the subject S. In addition, according to the gas inhalation mask 1 of Embodiment 1, the direction in which the tube connecting portion 122a and the tube axis TA illustrated in FIG. 1 face is rotatable in a range of 360°. Therefore, the tube can be routed more easily, and a force for deforming the mask body 11 is unlikely to transmit from the tube to the mask body 11.

In the tube attachment member 12, the hole of the inhalation flow path 122 is formed from the rear surface of the tube attachment member 12 to the inside of the tube connecting portion 122a. The inhalation flow path 122 has a hole provided perpendicular to the surface of the front locking portions 121a passing through the tube connecting portion 122a while being bent by 90° inside the tube attachment member 12. Accordingly, it is possible to prevent the vomitus or vomited liquid from entering the tube for supplying oxygen to some extent due to the bending of the inhalation flow path 122, even without the cover 123. However, in Embodiment 1, it is possible to reliably prevent the vomitus or the like from entering the tube due to the cover 123 and the bending of the inhalation flow path 122.

### Embodiment 2

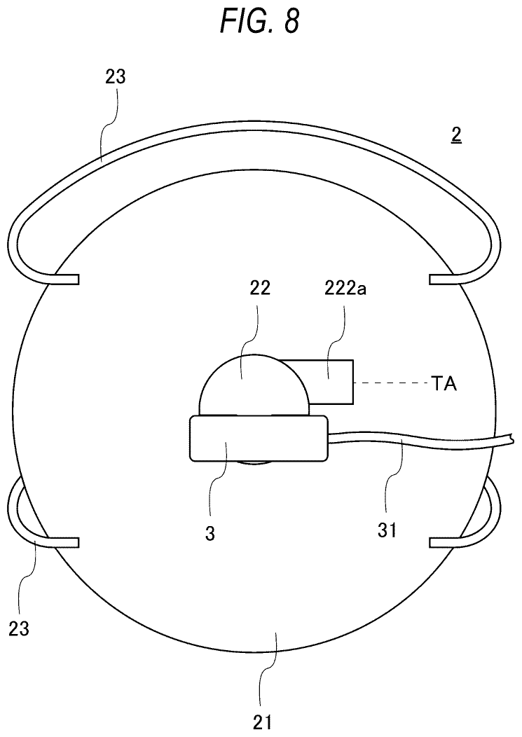

FIG. 8 illustrates a front view of a gas inhalation mask 2 in Embodiment 2. The gas inhalation mask 2 of Embodiment 2 is configured to measure the gas concentration by attaching a gas sensor 3. FIG. 8 illustrates a state in which the gas sensor 3 is attached to the gas inhalation mask 2. A tube attachment member 22 is attached to a mask body 21 to form the gas inhalation mask 2 of Embodiment 2, in the same or similar manner to Embodiment 1. Two elastic bands 23 are fixed to the mask body 21. The gas inhalation mask 2 can be fixed to the face of the subject S by stretching the two elastic bands 23 around to the back of the head of the subject S. The mask body 21 of Embodiment 2 is the same as the mask body 11 of Embodiment 1 illustrated in FIG. 3.

In the gas inhalation mask 2 illustrated in FIG. 8, the gas sensor 3 is attached to the tube attachment member 22. The gas sensor 3 of Embodiment 2 is configured to send, to a measuring instrument (not illustrated), an output corresponding to the amount of detected infrared, via a sensor wiring 31, and the carbon dioxide concentration is calculated and displayed on a display (not illustrated). In Embodiment 2, the sensor wiring 31 and a tube connecting portion 222a are directed to the same direction as illustrated in FIG. 8. The sensor wiring 31 and a tube for supplying gas (not illustrated) extend in the same direction in the vicinity of the gas inhalation mask 2.

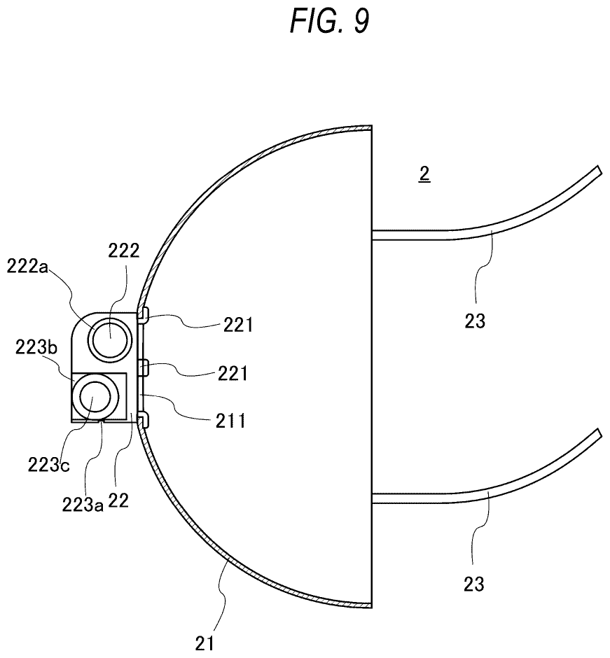

FIG. 9 illustrates a side view of the gas inhalation mask 2 of Embodiment 2. In FIG. 9, the mask body 21 is illustrated in a side cross-sectional view, and the tube attachment member 22 attached to the mask body 21 is illustrated in a side view. In FIG. 9, the gas sensor 3 is removed. The mask body 21 has a hemispherical shape. The tube attachment member 22 of Embodiment 2 may include an inhalation flow path 222 and an exhalation flow path 223 therein. The exhalation flow path 223 is configured to guide the exhaled gas out of the mask. The inhalation flow path 222 has a hole provided in the tube connecting portion 222a passing through the tube attachment member 22 and communicating with the inside of the mask body 21. The exhalation flow path 223 passes through the tube attachment member 22 from the inside of the mask body 21 and communicates with the exhalation discharge port 223a on the lateral side. A window 223c in FIG. 9 is provided in the middle of the exhalation flow path. The tube attachment member 22 of Embodiment 2 is formed of a high-molecular compound such as hard vinyl chloride or polyester, which are hard plastics.

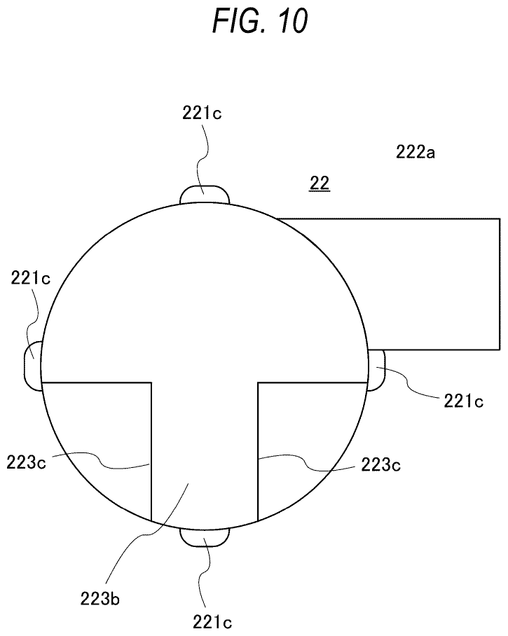

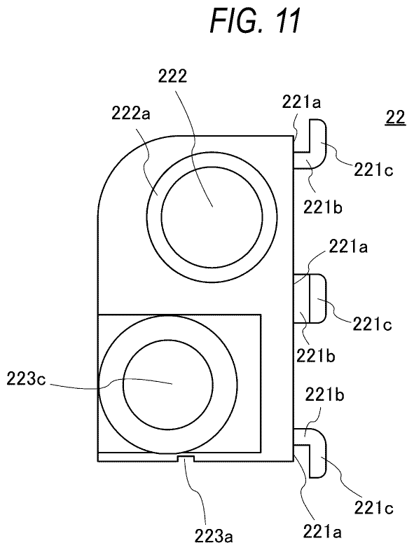

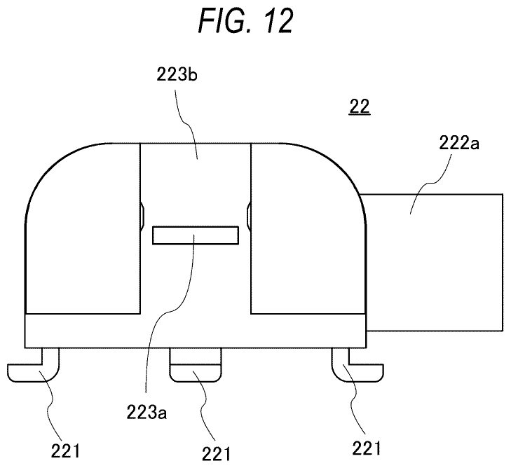

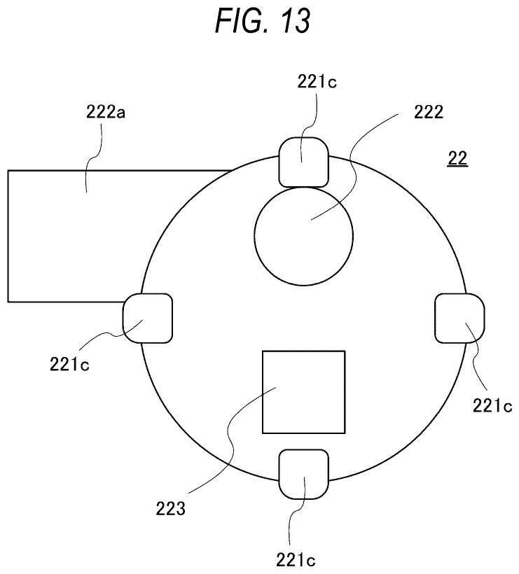

FIGS. 10 to 13 illustrate enlarged views of the tube attachment member 22 of the gas inhalation mask 2. FIG. 10 is a front view, FIG. 11 is a side view, FIG. 12 is a bottom view, and FIG. 13 is a rear view. The tube attachment member 22 is provided with locking portion connecting portions 221b at four positions on the rear side. The tip of each locking portion connecting portion 221b is provided with a rear locking portion 221c directed toward the vertical direction and the lateral direction of the tube attachment member 22. In the tube attachment member 22, four locking portion connecting portions 221b protrude rearward from front locking portions 221a on the rear side, which is right side in FIG. 11. The tip of each locking portion connecting portion 221b includes the rear locking portion 221c extending in the vertical direction and the lateral direction of the tube attachment member 22.

As illustrated in FIG. 11, the front side of each rear locking portion 221c is each front locking portion 221a. As illustrated in FIG. 9, the rear locking portions 221c are attached to the attachment opening 211 of the mask body 21 such that the rear locking portions 221c are positioned on the rear side of the attachment opening 211. Further, same or similar to Embodiment 1, the locking portion connecting portions 221b illustrated in FIG. 11 is located inside the circular attachment opening 211, and the periphery of the attachment opening 211 is sandwiched between the front locking portions 221a and the rear locking portions 221c, and the tube attachment member 22 is rotatably attached to the mask body 21. In FIG. 8, the tube connecting portion 222a of the tube attachment member 22 protrudes rightward, but the tube connecting portion 222a can be rotated in a range of 360°. The sensor wiring 31 is the same or similar.

In a case where the tube attachment member 22 is attached to the mask body 21, the tube attachment member 22 approaches the attachment opening 211 from the outer side of the mask body 21. The four rear locking portions 221c formed on the tube attachment member 22 are sequentially inserted into the attachment opening 211 of the mask body 21. In a case where the attachment is completed in this manner, the tube attachment member 22 is unlikely to be detached from the mask body 21 because the periphery of the attachment opening 211, which has strength to some extent, is sandwiched between the front locking portions 221a and the rear locking portions 221c at four positions. Further, the locking portion connecting portions 221b are positioned at four positions inside the circular attachment opening 211, the locking portion connecting portions 221b are configured to be moved along the inner side of the attachment opening 211, and the tube attachment member 22 is configured to be rotated in a range of 360°. In this manner, the direction in which the tube connecting portion 222a, the tube axis TA and the sensor wiring 31 illustrated in FIG. 8 face is configured to be rotated in a range of 360°.

In the rear surface of the tube attachment member 22 of Embodiment 2 illustrated in FIG. 13, the round hole is the inhalation flow path 222 and communicates with the hole provided in the tube connecting portion 222a. The oxygen supplied from the tube is supplied, into the mask body 21, from the inhalation flow path 222 illustrated in FIG. 13.

The rectangular hole illustrated in FIG. 13 is the inlet on the rear side of the exhalation flow path 223 provided in the tube attachment member 22. The exhalation flow path 223 illustrated in FIG. 13 is configured to discharge the exhaled gas out of the mask from the exhalation discharge port 223a in FIG. 12, which illustrates the lower surface of the tube attachment member 22. The exhalation flow path 223 configured to pass the exhaled gas is provided with, on both lateral sides, windows 223c through which light passes. One window 223c is illustrated in FIG. 11, and the other window 223c is provided at a position opposite to the window 223c illustrated in FIG. 11. The positions of the two windows 223c are illustrated in FIG. 10. The part of the exhalation flow path 223 provided with the windows 223c is a rectangular hole, and the windows 223c are provided in two walls facing each other.

The tube attachment member 22 including the inhalation flow path 222 and the exhalation flow path 223. In the tube attachment member 22, the hole of the inhalation flow path 222 is formed from the rear surface of the tube attachment member 22 to the inside of the tube connecting portion 222a. The inhalation flow path 222 has a hole provided perpendicular to the surface of the front locking portions 221a passing through the tube connecting portion 222a while being bent by 90° toward the lateral side in the tube attachment member 22. Although the inhalation flow path 222 does not include the cover as in Embodiment 1, it is possible to prevent the vomitus or vomited liquid from entering the tube for supplying oxygen due to the bending of the inhalation flow path 222. However, it is possible to reliably prevent the vomitus or the like from entering the tube in a case where a cover is provided as in Embodiment 1. The exhalation flow path 223 has a rectangular hole provided perpendicular to the surface of the front locking portions 221a being bent downward by 90° in the tube attachment member 22, passing between the windows 223c provided in a sensor attachment portion 223b, and being connected to the exhalation discharge port 223a facing downward. The exhalation flow path 223 does not include a cover.

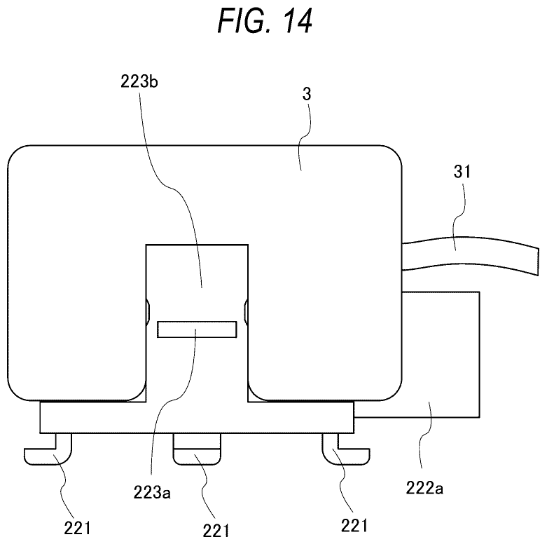

The gas sensor 3 illustrated in FIG. 8 may have a U shape. FIG. 14 is a bottom view of the tube attachment member 22 to which the gas sensor 3 is attached. The gas sensor 3 straddles the sensor attachment portion 223b through which the exhalation flow path 223 passes. One of the two branches of the gas sensor 3 includes an infrared light emitter inside one of the two branches. The other of the two branches of the gas sensor 3 includes an infrared light detecting element inside the other of the two branches. Infrared is irradiated toward one of the windows 223c, and the infrared passing through the exhalation flow path 223 is detected through the other window 223c. Since infrared of a specific wavelength is absorbed by carbon dioxide, the carbon dioxide concentration in the exhaled gas flowing through the exhalation flow path 223 can be detected based on the intensity of the detected infrared of the specific wavelength.

According to the gas inhalation mask 2 of Embodiment 2 as illustrated in FIGS. 8 and 9, the tube axis TA does not directly face toward the face of the subject S in a case where the mask body 21 is attached to the face of the subject S. Therefore, the force for deforming the mask body 21 is unlikely to be transmitted from the tube to the mask body 21. Further, the tube can be routed easily, in a case where the gas inhalation mask 2 is attached to the subject S. In addition, according to the gas inhalation mask 2 of Embodiment 2, the direction in which the tube connecting portion 222a and the tube axis face is configured to rotate in a range of 360°. Therefore, the tube can be routed more easily, and the force for deforming the mask body 21 is unlikely to transmit from the tube to the mask body 21.

### Embodiment 3

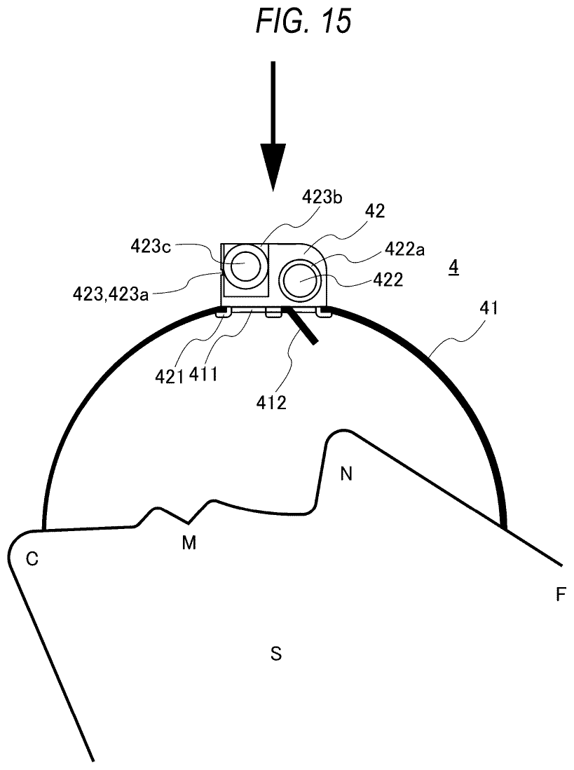

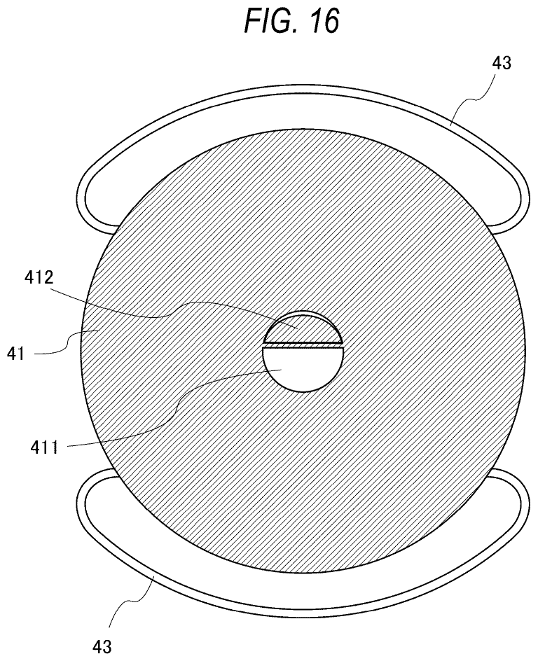

FIG. 15 illustrates a side view of a gas inhalation mask 4 of Embodiment 3 attached to the subject S. In FIG. 15, a mask body 41 is illustrated in a side cross-sectional view, and a tube attachment member 42 attached to the mask body 41 is illustrated in a side view. In FIG. 15, the gas inhalation mask 4 is attached to the face of the subject S facing upward. The elastic band 43 is not illustrated in FIG. 15, and is illustrated in FIG. 16 illustrating the mask body 41. The mask body 41 has a hemispherical shape. The tube attachment member 42 is the same or similar as the tube attachment member 22 of Embodiment 2. The tube attachment member 42 may include an inhalation flow path 422 and an exhalation flow path 423 (not illustrated) passing through inside the tube attachment member 42, a tube connecting portion 422a, an exhalation discharge port 423a, a sensor attachment portion 423b, and windows 423c. As illustrated in FIG. 15, the center of an attachment opening 411 is located between the nose N and the mouth M of the subject S, as viewed from the front indicated by the arrow in a case where the mask body 41 is attached to the subject S.

In Embodiment 3, a part of a cut-out portion remains in the attachment opening 411 of the mask body 41 to form a cover 412. FIG. 16 is a rear view of the mask body 41. A portion of the mask body 41 formed of thick paper is illustrated with diagonal lines. The mask body 41 is cut out in a semicircular shape at a portion slightly lower than the center portion to form the attachment opening 411. An arc-shaped slit is formed above the central portion, and the arc-shaped slit is configured to be folded toward the rear side of the mask body 41 to form the cover 412. The cover 412 is continuous to the outer mask body 41 in the vicinity of the fold. Since Embodiment 3 has the continuous portion, the tube attachment member 42 is not configured to be rotated.

According to the gas inhalation mask 4 of Embodiment 3 as illustrated in FIG. 15, the tube axis does not directly face toward the face of the subject S in a case where the mask body 41 is attached to the face of the subject S. Therefore, the force for deforming the mask body 41 is unlikely to transmit from the tube to the mask body 41. Further, the tube can be routed easily when attached to the subject S.

As illustrated in FIG. 15, the cover 412 provided to the mask body 41 is positioned between the position of the mouth of the subject S and the outlet of the inhalation flow path 422 provided in the tube attachment member 42, thereby functioning as a filter. The vomitus or vomited liquid from the mouth of the subject S is unlikely to enter the inhalation flow path 422 due to the cover 412. Therefore, the vomitus or vomited liquid can be prevented from entering the tube for supplying oxygen. In addition, the inhalation flow path 422 is bent by 90°, and the vomitus or the like is unlikely to reach the tube. The tube is not contaminated and thus does not need to be disposable, and can be reused to further reduce the disposal amount of plastic.

### Embodiment 4

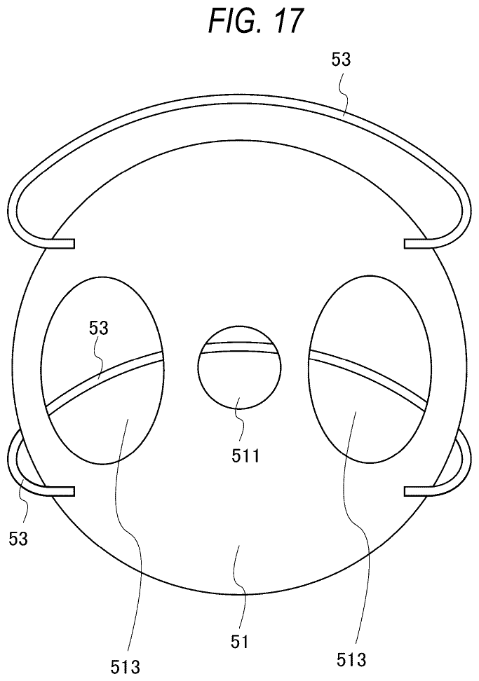

FIG. 17 is a front view of a mask body 51 according to Embodiment 4. The mask body 51 may have a circular attachment opening 511, and the tube attachment member 12 of Embodiment 1 or the tube attachment member 22 of Embodiment 2 can be rotatably attached. The mask body 51 has openings 513 in both lateral sides of the attachment opening 511. Two elastic bands 53 are fixed to the mask body 51.

In Embodiments 1 to 3, the mask body is formed of thick paper and has no transparency. Therefore, in a case where the gas inhalation mask using the mask body is attached to the subject S, the complexion or the like of the subject S cannot be observed. However, unlike Embodiments 1 to 3, the mask body 51 of Embodiment 4 illustrated in FIG. 17 is provided with openings 513 on both lateral sides. The face of the subject S inside the mask body 51 can be observed through the openings 513. Although the supplied oxygen leaks from the openings 513, the subject S can take oxygen at a high concentration since there is a portion covered with the mask body 51. In the case of using the tube attachment members 22 and 42 of Embodiments 2 and 3, the carbon dioxide concentration in the exhaled gas can be detected because a part of the exhaled gas enters the exhalation flow paths 223 and 423. In addition, it is generally recommended to blow in oxygen of 5 L/min or more in order to blow off the exhaled gas inside the mask, but in Embodiment 4, since the mask body is open, the re-inhalation amount of the exhaled gas is small, and oxygen of a constant flow rate can be administered.

### Embodiment 5

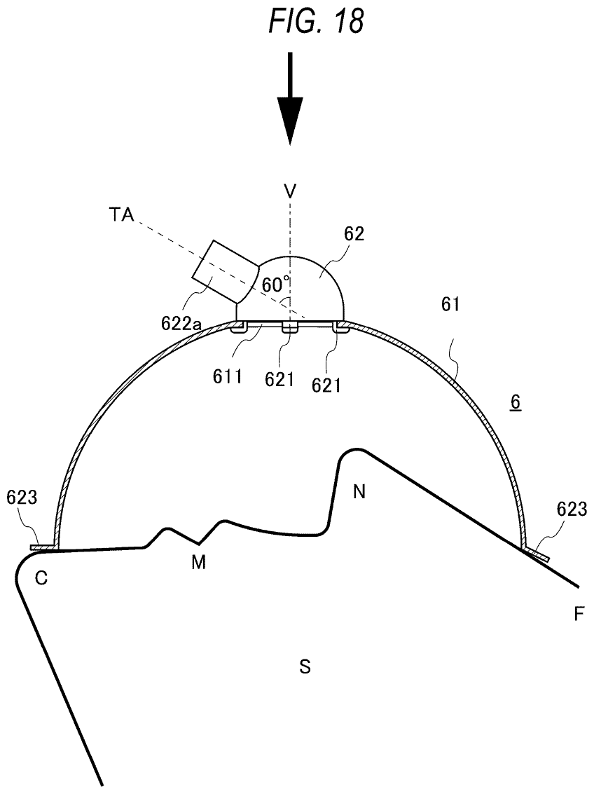

FIG. 18 illustrates a side view of a gas inhalation mask 6 in Embodiment 5 attached to the subject S. A mask body 61 is illustrated in a side cross section, and a tube attachment member 62 attached to the mask body 61 is illustrated in a side view. In FIG. 18, the gas inhalation mask 6 is attached to the face of the subject S facing upward. Same or similar to Embodiments 1 and 2, the tube attachment member 62 is rotatably attached to an attachment opening 611 of the mask body 61 by locking portions 621. In FIG. 18, the elastic bands are not illustrated. In Embodiment 5, a flange 623 is formed around the mask body 61. The flange 623 increases the width of the edge of the mask body 61 in contact with the face of the subject S.

As illustrated in FIG. 18, a tube connecting portion 622a of the tube attachment member 62 is inclined. An inhalation flow path 622 (not illustrated) is provided in the tube connecting portion 622a, same or similar to the other embodiments. In a case where the mask body 61 is attached to the subject S, the center of the attachment opening 611 is located between the nose N and the mouth M of the subject S, as viewed from the front indicated by the arrow. In Embodiment 5, the center of the attachment opening 611 coincides with the center of the mask body 61. The deviation between the center of the mask body 61 and the center of the attachment opening 611 is preferably within 10% of the width of the mask body 61 in both the longitudinal direction and the lateral direction, where the longitudinal direction is defined as a direction from the forehead F to chin C in a case where the gas inhalation mask 6 is attached to the face of the subject S, and the lateral direction is defined as the left-right direction of the face.

In FIG. 18, the tube axis TA which is the axis of the tube to be connected (not illustrated) is indicated by a dotted line, and the vertical direction V with respect to the attachment opening 611 is indicated by a one-dot chain line. As illustrated in FIG. 18, in the tube connecting portion 622a, the tube axis TA that is the axis of the tube to be attached is in a direction of 60° with respect to the vertical direction V of the attachment opening 611. Accordingly, the tube is configured to be connected to the tube connecting portion 622a in a direction shifted from the vertical direction V of the attachment opening 611. In Embodiment 1 and the like, the tube axis TA is in a direction of 90° with respect to the vertical direction V of the attachment opening 611. In Embodiment 5, the tube axis TA connected to the tube connecting portion 622a of the tube attachment member 62 does not directly face toward the face of the subject S in a case where the mask body 61 is attached to the subject S. Therefore, the tube can be easily routed in a case where the tube is connected to the tube connecting portion 622a. Further, a force is unlikely to be applied to the vicinity of the attachment opening 611 of the mask body 61, and the mask body 61 is unlikely to be deformed. In addition, according to the gas inhalation mask 6 of Embodiment 5, the direction in which the tube connecting portion 622a and the tube axis TA face is configured to be rotated. Therefore, the tube can be routed more easily, and a force for deforming the mask body 61 is unlikely to transmit from the tube to the mask body 61.

The periphery of the mask body 61 of Embodiment 5 is provided with the flange 623, which is a face contact piece oriented outward from the edge, to increase the width of the edge in contact with the face of the subject S. However, the area of the portion configured to contact the face of the subject S may be increased by providing a face contact piece directed inward from the edge on the entire circumference or a part of the entire circumference of the mask body. Further, the shape of the edge may be a curved surface along the shape of the face.

### Embodiment 6

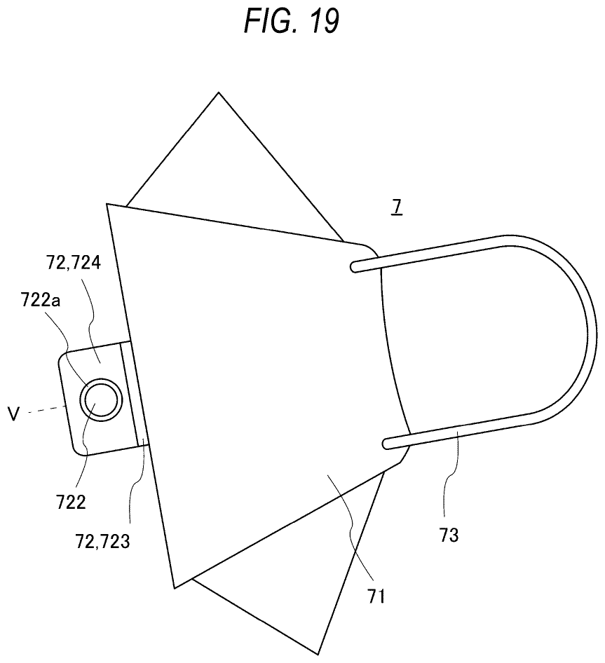

In the above embodiments, the mask body of the gas inhalation mask has a hemi-spherical shape, but a beak-shaped (diamond-shaped, tri-fold, or the like) mask may be used. FIG. 19 is a side view of a gas inhalation mask 7 in Embodiment 6. A mask body 71 and elastic bands 73 in the gas inhalation mask 7 are a beak-shaped mask. The elastic bands 73 are used over the ears of the subject S. FIG. 19 illustrates a state in which the tri-fold is opened. The mask body 71 is made of plastic fiber, and is mainly made of polypropylene nonwoven fabric.

In the gas inhalation mask 7 of Embodiment 6, a tube attachment member 72 adhere to the mask body 71. Same or similar to the tube attachment member 12 of Embodiment 1, an inhalation flow path 722 is bent by 90° from the hole of a tube connecting portion 722a toward the mask body 71, and is connected to the hole oriented toward the mask body 71. Unlike Embodiment 1 and the like, the mask body 71 is not provided with an attachment opening, and the fixed portion 723 of the tube attachment member 72 adhere to the mask body 71.

Since the mask body 71 is not provided with an attachment opening, the outlet of the inhalation flow path 722 in the tube attachment member 72 is closed by a part of the mask body 71. However, since the mask body 71 is a nonwoven fabric, the oxygen that has passed through the inhalation flow path 722 passes through the mask body 71 and is supplied to the rear side of the mask body 71. Thus, in Embodiment 6, a part of the mask body 71 serves as a cover. Further, the vomitus or vomited liquid from the mouth of the subject S does not enter the inhalation flow path 722 by the cover which is a part of the mask body 71. The vomitus does not enter the inhalation flow path 722 either in a case where the supply of oxygen from the inhalation flow path 722 is stopped. Therefore, the vomitus or vomited liquid can be prevented from entering the tube connected to the tube connecting portion 722a for supplying oxygen. The tube is not contaminated and thus does not need to be disposable, and can be reused to further reduce the disposal amount of plastic.

The tube attachment member 72 is rotatably connected between the fixed portion 723 and a rotatable portion 724. Therefore, the direction in which the tube connecting portion 722a protrudes and the direction of the tube axis TA are configured to be rotated in a range of 360°. In the case of Embodiment 6, the tube axis TA, which is the axis of the tube, is in a direction of 90° with respect to the vertical direction V of the attachment surface of the tube attachment member 72 to the mask body 71, but is in a direction not directly face toward the face of the subject S.

According to the gas inhalation mask 7 of Embodiment 6 as illustrated in FIG. 19, the tube axis does not directly face toward the face of the subject S in a case where the mask body 71 is attached to the face of the subject S. Therefore, the force for deforming the mask body 71 is unlikely to transmit from the tube to the mask body 71. Further, the tube can be routed easily when attached to the subject S. In addition, according to the gas inhalation mask 7 of Embodiment 6, the direction in which the tube connecting portion 722a and the tube axis face is configured to be rotated in a range of 360°. Therefore, the tube can be routed more easily, and a force for deforming the mask body 71 is unlikely to transmit from the tube to the mask body 71.

In the above embodiments, the mask body of the gas inhalation mask is a hemi-spherical or beak-shaped (diamond-shaped) mask, but may have another shape such as a shape along the face. However, in order to effectively use the gas supplied at the time of exhalation by the subject S, the space between the mask body and the face of the general subject S is preferably 30 cc or more and 200 cc or less, more preferably 80 cc or more and 150 cc or less.

In Embodiments 1 to 5 of the presently disclosed subject matter, the mask body is formed of paper made of natural fiber as a raw material, and the tube attachment member is formed of hard vinyl chloride which is hard plastic. However, the mask body may be formed of fabric or nonwoven fabric as in Embodiment 6. The paper, nonwoven fabric, or fabric may be partially or entirely formed of plastic fibers as in Embodiment 6. The nonwoven fabric or fabric preferably has a hardness that can maintain a space on the rear side of the mask body. The nonwoven fabric or the fabric may or may not be air-permeable.

Examples of the raw material of the fibers of the paper, nonwoven fabric, or fabric used for the mask body include polyester, polypropylene, cotton, wood pulp, and the like. A mask body of the related art is manufactured by molding a high-molecular compound such as soft vinyl chloride or polyester. The material melted during plastic molding is put into a mold and hardened, which causes a large thickness of the mask body. This increases the weight of the used plastic. The nonwoven fabric or fabric can be formed thin without molding as in the related art, even in a case where polyester fibers or the like are used. Since the paper, nonwoven fabric or fabric is made of fibers, strength and hardness required for the mask body can be obtained even in a case where the mask body is thin. Even in a case where the entire mask body is made of plastic, the mask body can be made with plastic of a weight smaller than that of the plastic mask body of the related art. In a case where the used fibers are obtained by mixing natural fibers into plastic fibers or are all natural fibers, a mask body having a smaller environmental burden can be formed.

In the embodiments, oxygen is supplied to the subject, but other gases may be supplied. The tube attachment member may be formed of metal or the like other than plastic. Hard plastic is used in the embodiment because the tube attachment member is smaller than the mask body, but the environmental burden can be further reduced by using a material derived from a natural material, such as a material obtained by solidifying sawdust with an adhesive.

As described in the embodiment, the tube attachment member is configured such that the tube axis, which is the axis of the tube connected to the tube connecting portion, does not directly face toward the face of the subject in a case where the mask body is attached to the subject. The tube is connected to the tube connecting portion in a direction shifted from the vertical direction with respect to the attachment opening. The angle between the tube axis and the vertical direction with respect to the attachment opening or the attachment surface of the tube attachment member is 90° in Embodiments 1 to 4 and 6, and is 60° in Embodiment 5. The tube axis is preferably 45° or more and 100° or less, and more preferably 60° or more and 95° or less with respect to the vertical direction of the attachment opening or the attachment surface of the tube attachment member.

In Embodiments 1, 2, 4 and 5, the attachment opening has a circular shape, and the tube attachment member is attached to the attachment opening of the mask body to be rotated in a range of 360° by being held by the front locking portions, the locking portion connecting portions, and the rear locking portions. However, the rotatable range may be set to a part of a range of 360° by changing the shape of the attachment opening or the like. In addition, as in Embodiment 3, the tube attachment member may be attached in a non-rotatable manner depending on the shape of the attachment opening. The tube attachment member may be fixed to the mask body around the attachment opening. The tube attachment member may be firmly fixed by strongly sandwiching the edge of the attachment opening with the configuration of the tube attachment member.

In Embodiment 1, the cover 123 made of a mesh material is provided at the outlet of the inhalation flow path 122. In Embodiment 3, a part cutout from the mask body 41 when forming the attachment opening 411 serves as the cover 412. However, another member may be provided between the position of the mouth of the subject S and the inhalation flow path to serve as the cover. Further, an air-permeable member may be attached to the inhalation flow path to serve as the cover. By providing the cover, in a case where the supply of the gas from the inhalation flow path is stopped, the vomitus or vomited liquid can be filtered out from the opening of the subject to be prevented from entering the tube for supplying the gas. The vomitus and the vomited liquid is particularly likely to reach the tube particularly in a case where the gas supply from the tube is stopped. Since the tube is not contaminated due to the presence of the cover, the plastic tube does not need to be disposable, which can reduce the disposal amount of the plastic.

The mainstream method of detecting the carbon dioxide concentration by directly measuring the exhaled gas is used in Embodiments 2 and 3, and the sidestream method of detecting the carbon dioxide concentration by aspirating a part of the exhaled gas may be used.

The specific configuration is not limited to the embodiments, and modifications and the like of the design without departing from the gist of the presently disclosed subject matter are also included in the presently disclosed subject matter. The embodiments described above can be combined by using techniques of one another as long as without particular contradiction or problem in the object, configuration, and the like.

This application claims priority to Japanese Patent Application No. 2022-158571 filed on Sep. 30, 2022, the entire content of which is incorporated herein by reference.

### Industrial Applicability

According to the presently disclosed subject matter, it is possible to provide a gas inhalation mask that is environmentally friendly and has sufficient strength.

1. A gas inhalation mask comprising:
a mask body formed of paper, nonwoven fabric, or fabric; and
a tube attachment member,
wherein the tube attachment member is attached to an attachment opening provided in the mask body, and
in a case where the mask body is attached to a subject, an axis of a tube does not directly face toward a face of the subject, the tube being connected to a tube connecting portion of the tube attachment member.

2. The gas inhalation mask according to claim 1,
wherein the tube attachment member is configured to be rotated in a range of 360° or in a part of the range of 360°, the tube attachment member being attached to the attachment opening of the mask body.

3. The gas inhalation mask according to claim 1,
wherein the tube attachment member includes a front locking portion and a rear locking portion, and
the tube attachment member is attached to the attachment opening in a state where a periphery of the attachment opening of the mask body is sandwiched between the front locking portion and the rear locking portion.

4. The gas inhalation mask according to claim 1,
wherein the tube attachment member adhere to the mask body.

5. The gas inhalation mask according to claim 1,
wherein, in a case where the mask body is attached to the subject, a center of the attachment opening is located between a nose and a mouth of the subject.

6. The gas inhalation mask according to claim 1,
wherein the attachment opening is partially or entirely covered with a cover, and
the cover is configured to filter vomitus or vomited liquid of the subject.

7. The gas inhalation mask according to claim 1,
wherein the mask body has an opening in a portion other than the attachment opening, and
the face of the subject inside the mask body is observed through the opening.

8. The gas inhalation mask according to claim 1,
wherein the tube attachment member includes an inhalation flow path and an exhalation flow path,
the inhalation flow path is connected to a hole of the tube connecting portion, and
the exhalation flow path is configured to guide exhaled gas out of the mask.

9. The gas inhalation mask according to claim 8,
wherein the exhalation flow path includes, on both lateral sides, windows through which light for measurement passes.

10. The gas inhalation mask according to claim 1,
wherein a raw material of a fiber used for the paper, the nonwoven fabric, or the fabric includes polyester, polypropylene, cotton, or wood pulp.
